(19) United States
(12) Patent Application Publication
(10) Pub. No.: US 2010/0312123 A1
(43) Pub. Date: Dec. 9, 2010

(54) CARDIAC MEASUREMENT SYSTEM AND METHOD

(75) Inventor: Robert Allan Phillips, New South Wales (AU)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(73) Assignee: USCOM LIMITED, Sydney, New South Wales (AU)

(21) Appl. No.: 12/301,979

(22) PCT Filed: May 22, 2007
(86) PCT No.: PCT/AU2007/000701

§ 371 Date: Aug. 13, 2010

(30) Foreign Application Priority Data
May 22, 2006 (AU) 2006902751
Mar. 20, 2007 (AU) 2007901454

(51) Int. Cl. A61B 5/021

(52) U.S. Cl. 600/485

(57) ABSTRACT

A method of monitoring blood flows through the body includes performing a substantially continuous blood pressure monitoring of a vessel at a first predetermined location to obtain a substantially continuous blood pressure signal; performing blood flow monitoring of a vessel at a first predetermined location to obtain a substantially continuous blood flow signal; providing an updated cardiac measure of said patient by continuously processing the flow signal and the pressure signal.

### RELATED APPLICATIONS

This is a U.S. National Stage application which claims priority to PCT Application No. PCT/AU2007/000701, filed May 22, 2007, Australian Application No. 2006902751 filed May 22, 2006, and Australian Application No. 2007901454 filed Mar. 20, 2007, the disclosures of which are hereby incorporated herein by reference in their entirety.

### TECHNICAL HELD

The present invention relates to ultrasound monitoring and in particular to a system and method for measuring the cardiac output power of a patient's heart.

The invention has been developed primarily for use as Doppler monitoring system and provides real-time measures of cardiac power of a patient derived from continuous wave Doppler ultrasound and blood pressure measurements and will be described hereinafter with reference to this application. However, it will be appreciated that the invention is not limited to this particular field of use.

### BACKGROUND

Any discussion of the prior art throughout the specification should in no way be considered as an admission that such prior art is widely known or forms part of the common general knowledge in the field.

Cardiac performance is a function of the fluid flowing into the heart. The determinants of flow include, the preload, the performance of the heart (contractility and relaxation), and the afterload or systemic vascular resistance (SVR). However it is hypothesised that these parameters, while useful, do not completely describe cardiac function.

The concept of Cardiac Power Output (CPO) and Stroke Work (SW) have been advanced as possible improvements to the basic assessment methods that can be derived from stroke volume (SV) and cardiac output (CO) values.

Cardiac Power Output (CPO) is defined as the product of mean Blood Pressure (BP) and Cardiac Output (CO).

Stroke Work (SW) is defined as the amount of energy that is given to the blood in a single heart stroke, With Cardiac Work (CW) further defined as the product of SW and the patient's heart rate.

CO and SV, which form the basis for calculating measures of CPO and SW, are typically measured using instantaneous single measures from echocardiography or invasive catheter derived measures. The most common method being Pulmonary Artery Thermodilution (or Transpulmonary Thermodilution), which is an invasive procedure requiring the use of a pulmonary artery catheter (PAC) also known as the Swan-Ganz thermodilution catheter and provides a measure of right heart blood pressures. Using the PAC thermodilution CO can be measured, from which the CPO and SW can be derived.

These measures are typically made at one time and may take up to an hour to measure using invasive catheter methods. During this period the values of the measured parameters may change along with the patients condition. As CPO and SW vary beat to beat, reflecting changes in physiology and pathophysiology, a method of measuring SV and CO in real time for calculating CPO and SW will substantially improves these previously described methods.

Hence there is a need in the art for providing real-time, preferably non-invasive, measurement of the right and left ventricular cardiac power and cardiac work.

### SUMMARY OF THE INVENTION

It is an object of the invention in its preferred form to provide methods and apparatus for measurement of Cardiac Output (CO) and Stroke Volume (SV) in real-time for use in calculating a real-time measurement of cardiac power (CPO) and cardiac work (CW).

In accordance with a first aspect of the present invention, there is provided a method comprising the steps of: performing a substantially continuous blood pressure monitoring at a first predetermined location to obtain a substantially continuous blood pressure signal; obtaining a calibrated measurement of cardiac output from a direct cardiac output device; and produce a cardiac approximation to blood flowing through the body by utilising the calibrated measurement to manipulate the substantially continuous blood pressure signal.

In an example embodiment the calibrated measurement may be utilised to transform the substantially continuous blood pressure signal to produce a cardiac approximation to blood flowing through the body by the steps of:

calculating a transfer function for transforming the substantially continuous blood pressure signal to produce a cardiac approximation to blood flowing through the body; and applying the transfer function to the substantially continuous blood pressure signal to produce the cardiac approximation to blood flowing through the body.

In an embodiment the transfer function is a scalar value. In a further embodiment the calculation of a transfer function is preferably automatically and calculated repeatedly.

In another embodiment, manipulating the substantially blood pulse pressure signal is performed for each obtained calibrated measurement.

An example setup produces a cardiac approximation to blood flowing through the body and is substantially continuous. In another embodiment, cardiac approximation to blood flowing though the body is produced in real time.

An example embodiment for providing a measurement of cardiac power of a patient comprises:

a transducer device for providing a monitoring of flows within the patient and providing a flow signal;

a blood pressure monitor for providing a blood pressure signal;

a processing unit adapted to receive the flow signal and the blood pressure signal and adapted to process the flow signal and the pressure signal so as to provide a measurement of the cardiac power of the patient.

The transducer device may be a Doppler ultrasonic transducer. In an example embodiment, the transducer device and the pressure monitor each continuously provide respective flow and pressure signals to provide a continuous measurement of the cardiac power.

The measurement of cardiac power may be provided in real-time.

An example embodiment for providing a real-time measurement of cardiac power of a patient comprises:

continuously monitoring blood flows in a patient to provide a flow signal;

continuously monitoring blood pressure to provide a pressure signal;

continuously processing the flow signal and the pressure signal to provide the cardiac power measurement of the patient.

The flow signal may be provided by a Doppler ultrasonic transducer and the pressure signal may be provided by a blood pressure monitor.

An example embodiment provides a method of monitoring blood flow through the body comprising the steps of:

performing a substantially continuous blood pressure monitoring of a vessel at a first predetermined location to obtain a substantially continuous blood pressure waveform;

obtaining a calibrated measurement of cardiac output from a direct cardiac output device; and

producing a cardiac approximation to blood flowing through the body by utilising the calibrated measurement to manipulate the substantially continuous blood pressure waveform.

In an example embodiment, utilising the calibrated measurement to manipulate the substantially continuous blood pressure signal is repeated automatically for each obtained calibrated measurement. In one embodiment the obtaining of a calibrated measurement is intermittent.

In an example embodiment, utilising the calibrated measurement to manipulate the substantially continuous blood pressure signal comprises the steps of:

calculating a transfer function for transforming the substantially continuous blood pressure signal to produce a cardiac approximation to blood flowing through the body; and

producing the cardiac approximation to blood flowing through the body by applying the transfer function to the substantially continuous blood pressure waveform.

In another example, the transfer function is automatically repeatedly calculated. In one embodiment this transfer function is a scalar value.

In a further example, the cardiac approximation to blood flowing through the body is substantially continuous. This cardiac approximation to blood flowing through the body is more preferably produced in real-time.

In a further example, the substantially continuous blood pressure monitoring is a non-invasive measurement. This non-invasive measurement is preferably performed by a tonometer or a sphygmomanometer. Alternatively the substantially continuous blood pressure monitoring is performed by a blood oximeter.

Alternatively, the substantially continuous blood pressure monitoring is an invasive measurement. This measurement may be performed by a manometer tipped catheter.

In an embodiment the substantially continuous blood pressure signal is tonometrically generated.

In another embodiment the substantially continuous blood pressure signal is Doppler generated from a peripheral vessel.

In an example embodiment, the direct cardiac output device comprises a USCOM device. Alternatively, the direct cardiac output device comprises an ultrasonic transcutaneous monitoring device.

Another embodiment provides a device for monitoring blood flows through the body, the device configured perform the method according to any of the preceding methods to produce a cardiac approximation to blood flowing through the body.

These methods and devices provide an improvement over current methods, by providing relative improvements to accuracy or safety, and by providing a real time celebrated cardiac output measurement waveform with reduced relative invasiveness.

### BRIEF DESCRIPTION OF THE DRAWINGS

An embodiment of the invention will now be described, by way of example only, with reference to the accompanying drawings in which:

FIG. 1 illustrates a simplified circulatory diagram demonstrating the relationship of the heart to the systemic and pulmonary circulation and the venous and arterial circulation;

FIG. 2 is a perspective view of an arrangement of the invention when utilised to monitor a patient according to the invention;

FIG. 3 illustrates pressure and flow signals at different points in the circulation, with the signal dependent on the diameter of the vessel and distance from the generator (heart), and the compliance of the vascular system (Mills, Cardiovasc Res 1970; 4:40-7);

FIG. 4 illustrates oximetric signals demonstrating pulsatile changes associated with changes in oxygen saturation;

FIG. 5 illustrates continuous wave Doppler signals demonstrating pulsatile changes associated with changes in SV, and quantitative values of flow;

FIG. 6 shows a schematic of a device according to an embodiment of the invention; and

FIG. 7 is a functional block diagram of an example embodiment of the invention.

FIG. 8 shows a flowchart of method according to an embodiment; and

FIG. 9 illustrates the step of combining the USCOM measurement with the blood pressure signal measurement.

### DETAILED DESCRIPTION

Monitoring of cardiovascular performance is important in many clinical environments. The circulation functions to transport oxygen and substrates to the tissues and returning the cell wastes for excretion. Blood is the transport medium of the circulation and caries the oxygen and substrates around a closed system within the body.

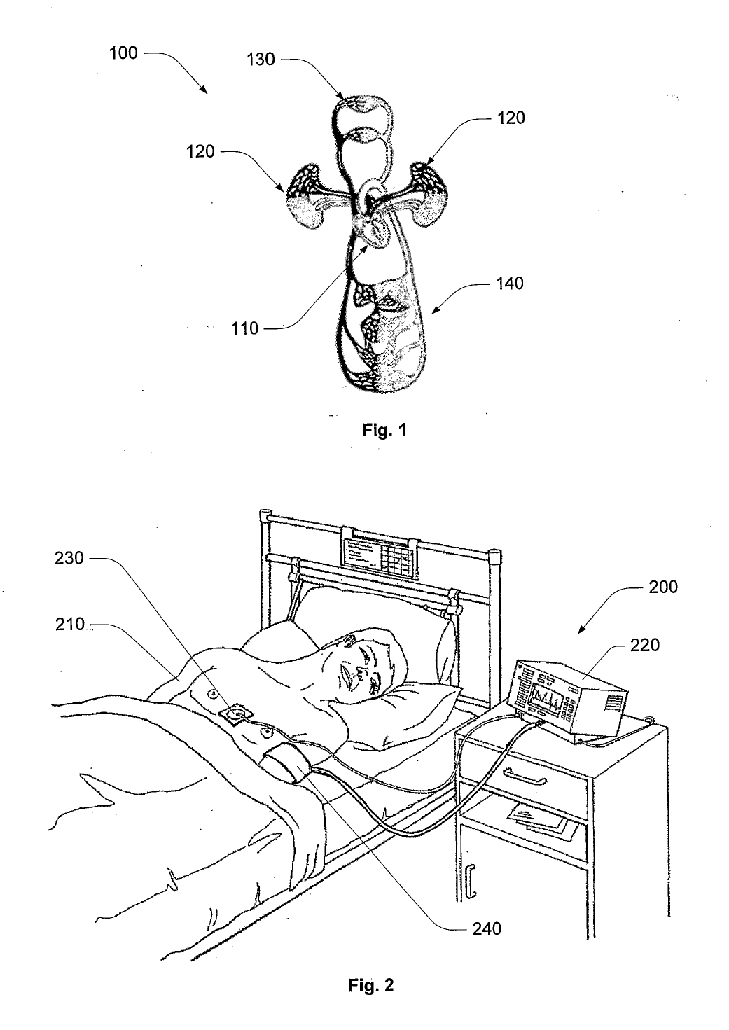

Referring initially to FIG. 1 of the drawings, this circulatory system 100 consists of ramifying arterial network originating as the aorta from the heart 110 and eventually dividing in to the smallest arterial units, the arterioles, and capillaries where the oxygen exchange takes place. The blood then returns from the small veins or venules which successively unite to form larger veins, until the two major veins, the inferior and superior vena cava unite in the right atrium of the heart. This is the systemic circulation. From the right side of the heart, blood travels into the lungs 120 where the dc-oxygenated blood is re-oxygenated for its transport back to the systemic circulation and the cells and tissues of the body such as the head 130 and lower body 140.

The blood is moved around this system primarily by the force of the heart, although the arteries and veins, muscular walled cylinders, also actively change resistance to the flow of blood thus changing the load on the heart. The heart functions as the pump in this system and the cardiac muscles rhythmically contract and relax changing the pressures within the chambers of the heart and driving the blood across the pressure gradients created by these contractions. The heart pumps into the vascular system against a resistance known as the afterload or systemic vascular resistance or the vascular compliance, which regulates the load on the heart.

Additionally, the heart is filled by venous blood returning to the heart, and this is often referred to as the preload and also related, via the Frank Starling mechanism, for the force of contraction of the heart. The interplay of these three dynamic entities modulates the circulation and ensures that oxygenated blood is delivered to the cells and de-oxygenated blood is returned to the lungs for oxygenation. The interplay of the preload, the cardiac function, and the afterload is fundamental to the efficiency of the circulation and is regulated on a beat to beat basis.

As the heart contracts the pressure in the left ventricle rises, the aortic valve opens and the pressure wave generated by the contraction of the ventricle is transmitted into the arterial tree. This pressure wave losses its energy the further it is from the generated source. However at any point in the vascular system, the greater the contraction of the heart, the greater will be the pressure peak and it is possible to monitor the blood pressure, or equivalently pulse pressure, at any point in the circulation using a number of invasive or non-invasive methods. This changing pressure is the blood pressure and can be monitored to assess cardiac function.

These fluxing pressures can be monitored at a relatively subjective level using digital measure of the pulse or as a quantitative measure using cuff or sphygmomanometric measurement of blood pressure. When the heart function is poor, the arterial pulse signals are diminished, while during increased output they are elevated. While the use of arterial pulse pressures to monitor cardiac function is established, it is clear that the pulse is a function of both the cardiac contraction and the vascular function including compliance, so that a strong pulse may be felt when cardiac function is poor but the vessels are constricted.

Referring to FIG. 2, there is provided an example embodiment of a system 200 for providing a measure of cardiac power of a patient 210. The system comprises a processing unit 220 adapted to receive and process a flow signal and the pressure signal for providing a measure of the cardiac power of the patient.

A transducer device 230 is used for monitoring flows within the patient and providing the flow signal. A blood pressure sensor 240 is used for providing the pressure signal.

In this example embodiment, the transducer device is in the form of a continuous wave (CW) Doppler ultrasound transducer 230, having a typical operating frequency range of 1.0 to 3.0 MHz. This transducer element can be strapped to the left ventricular apical intercostal window, the left parasternal intercostal window or the suprasternal notch for monitoring transvalvular blood flows in the heart. The transducer is typically fixed in place with an adhesive sheet or tape, and a belt. The transducer monitors flows within the patient and is coupled to the computer signal processing unit 220 for providing the flow signal.

In this example embodiment, the blood pressure sensor 240 is in the form of a non-invasive sphygmomanometric source comprising a pressure gauge and a rubber cuff that wraps around the upper arm of the patient. In another embodiments, the blood pressure sensor comprises of an intra-arterial catheter. It is preferred that the blood pressure sensor 540 monitors the patient's blood pressure continuously and produces a pressure signal corresponding to the patient's systolic and diastolic blood pressure. The blood pressure sensor is coupled to the computer signal processing unit 220 for providing the pressure signal.

There are a number of methods by which these blood pressure signals can be converted to a quantitative measure of cardiovascular function.

Essentially there are two main approaches including:
Invasive: arterial blood pressure monitoring using a manometer tipped catheter and positioning the transducer in the mid vessel so that flow dynamics are stable and representative. The smaller the artery the more noise from the measurement and the less accurate the signal, so the abdominal aorta is preferred however the internal iliac and radial artery can be accessed, and application in children is generally inaccurate and, due to invasiveness, undesirable.
Non-invasive: where a tonometer, sphygmomanometer or Doppler is applied at some point to sense the changes in blood pressure reflected in the small changes in wall pressure of an accessible vessel. This is usually at the medial cubital or radial arteries, however carotid, femoral, or peripheral pulses can also be measured,

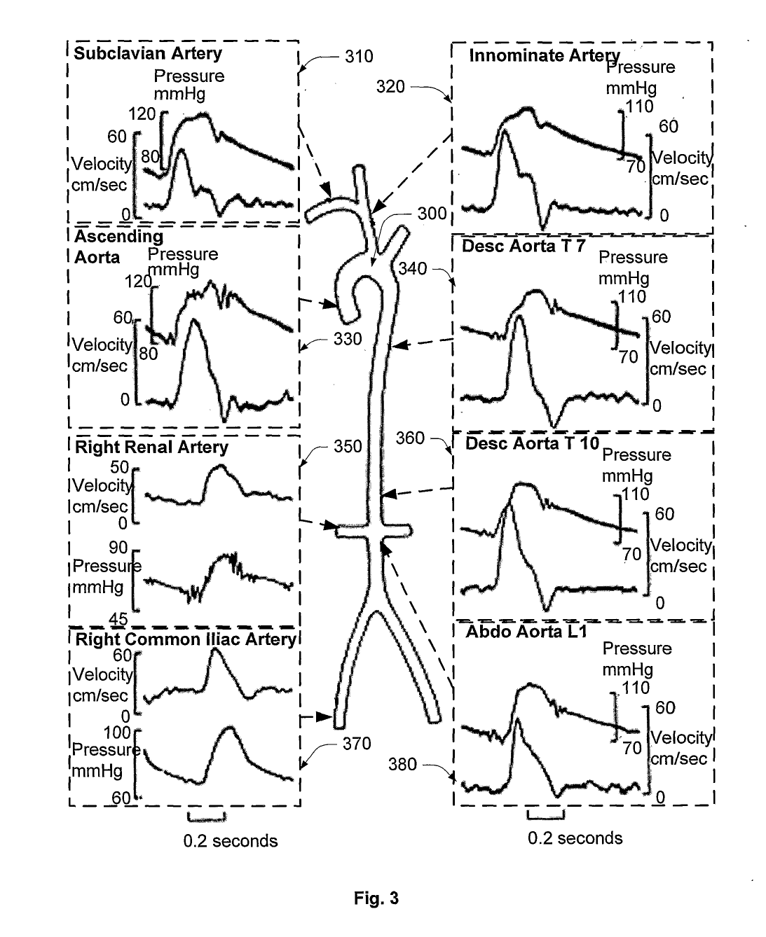

Each of these methods provides a simple blood pressure, or equivalently pulse pressure, signal indicating the sum of the cardiac and vascular performance. FIG. 3 illustrates example pressure and flow signals at different points in the circulation illustrated by a simplified arterial system 300, including the subclavian artery 310, innominate artery 320, ascending aorta 330, descending aorta T7 24, right renal artery 350, descending aorta T10 260, right common iliac artery 370 and abdo aorta L1 380 (Mills, Cardiovasc Res 1970; 4:40-7). These signals are dependent on the diameter of the vessel and distance from the generator (heart), and the compliance of the vascular system.

However in clinical practice it is preferred to understand both the cardiac and the vascular function individually as therapies are usually discrete and specific. For example, inotropes are used to stimulate cardiac contraction, while vasodilators relax the arterial vessel walls, dilating the artery, and reducing the vascular resistance, which in turn increases cardiac flow.

A simple measure of the blood pressure could be converted via a transfer function of constant (k) to calculate a quantitative cardiac output measurement, meaning that the blood pressure could be monitored as cardiac output. However, a limitation in this method is that blood pressure is a regulated variable, and as cardiac output drops as in disease, the arteries contract to maintain blood pressure and perfusion and as a result the method may monitor well in normal subjects, but is inaccurate in altered normal physiology or disease. Therefore the relationship between blood pressure and cardiac output varies.

Only over short periods of time, where the blood pressure signal was calibrated to a cardiac output measurement method, can monitoring blood pressure reflect changes in cardiac output. With an arterial manometer inserted and a blood pressure signal acquired, the blood pressure signal can be calibrated to an invasive transpulmonary thermodilution measurement of cardiac output. This calibration is recommended to be performed 8 hourly in stable monitoring, or whenever therapy is changed, the patient's position is changed, a therapy is introduced etc. Preferably the blood pressure signal should be calibrated beat to beat.

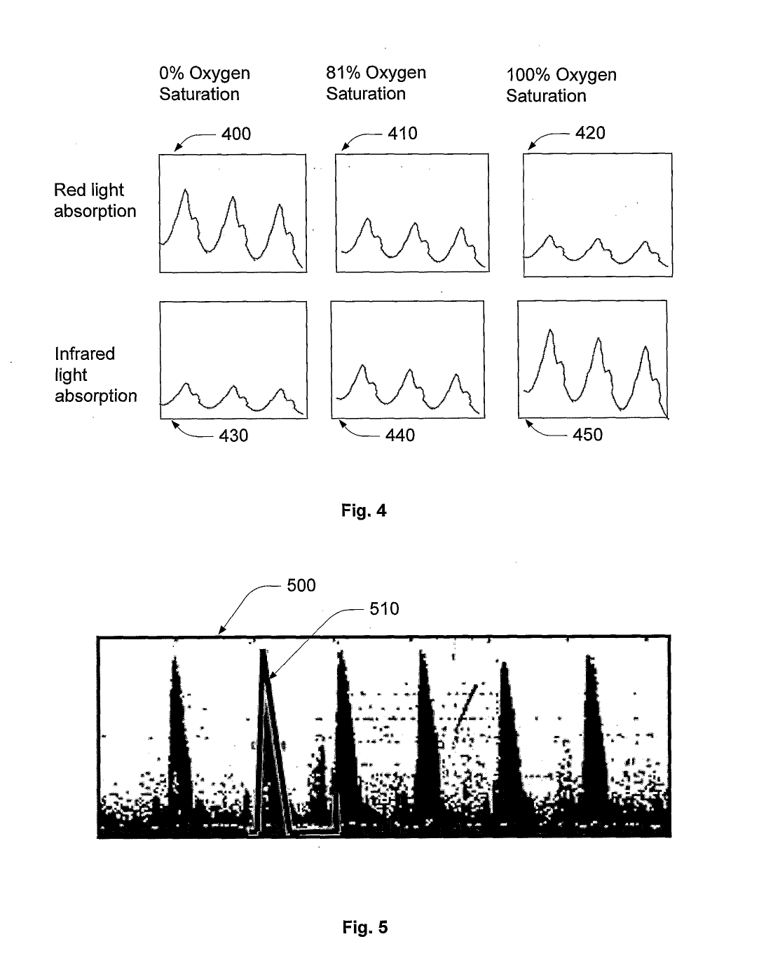

FIG. 4 illustrates oximetric signals measured using red-light absorption (e.g. 400 410 and 420) and infrared light absorption (e.g. 420 430 and 440) techniques, demonstrating pulsatile changes associated with changes in oxygen saturation associated with oxygen delivery, a product of blood oxygen saturation and cardiac output. Oximetric measurements can be made at any point in the peripheral circulation such as the finger, toe, tongue, ear and forehead.

In this embodiment, as shown in FIG. 2, the transducer device 230 is a CW Doppler ultrasonic transducer that has been adapted for use as a heart monitoring device. CW Doppler is a well evaluated and a routine echocardiographic method of quantifying cardiac output with low inter and intra observational variability.

The an embodiment of a system 200, as shown in FIG. 2, the CW Doppler can monitor both right and left ventriculo-arterial flow and can therefore measure CPO and SW of both the right and left ventricle. A pulmonary artery catheter, as it is lodged in the pulmonary artery, can only measure the flow across the pulmonary artery and so can only determine right ventricular CPO and SW.

U.S. Pat. No. 6,565,513 entitled “Ultrasonic Cardiac Output Monitor”, the contents of which are incorporated herein, measures this cardiac output accurately and non-invasively but requires a manual signal acquisition process to be carried out. Herein after referred to as the USCOM device. The USCOM device being available from USCOM limited of Sydney, Australia.

Direct measurement of transpulmonary flow can be achieved by applying a small CW Doppler transducer with an adherent gel coupling layer to the surface of the skin at the left parasternal acoustic window; adjacent to the sternum in an intercostal interspace, while the transaortic flow can be detected from the intercostal space associated with the palpable ventricular apex beat or from the suprasternal notch. The transducer can be fixed in place with adhesive tape or sheet and or a transthoracic belt utilising a thin gel coupling layer to ensure transducer skin contact.

FIG. 5 illustrates a CW Doppler signals 500 demonstrating pulsatile changes associated with changes in SV, and quantitative values of flow. Each pulse, e.g. 510, corresponds to the blood flow during a single heart beat.

A preferred embodiment utilises separate cardiac output monitoring to calibrate blood pressure signals to a standard base, thereby providing a calibrated continuous time signal of cardiac output.

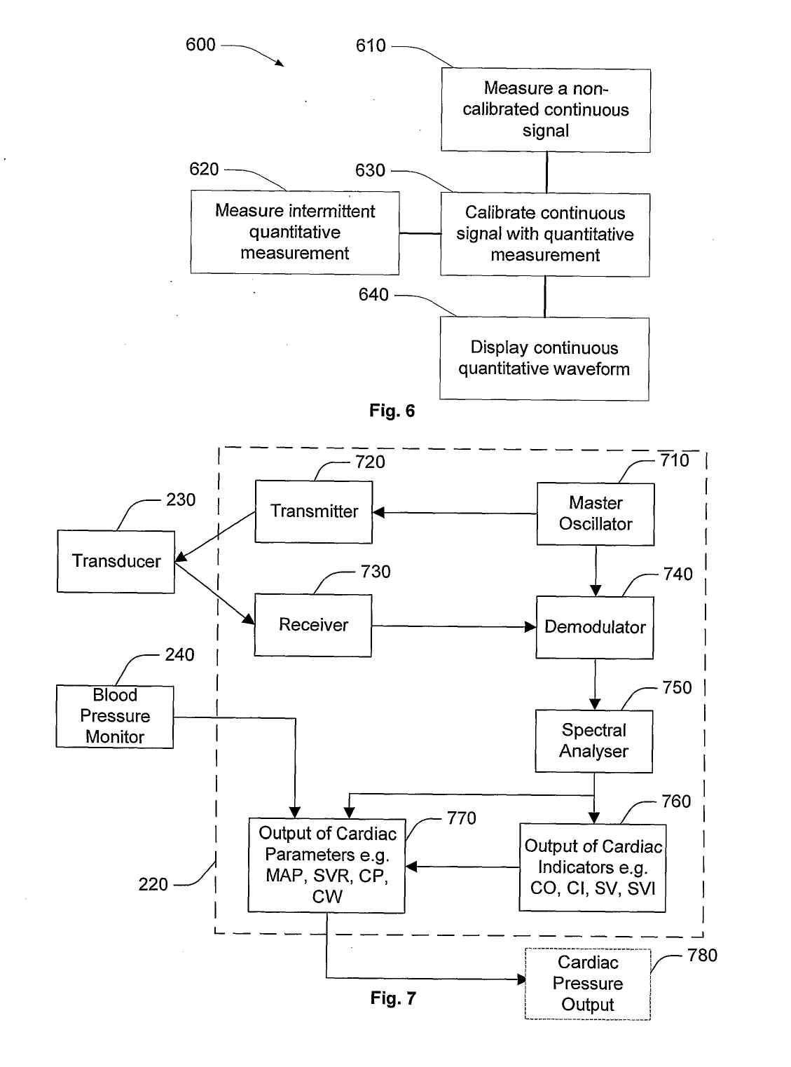

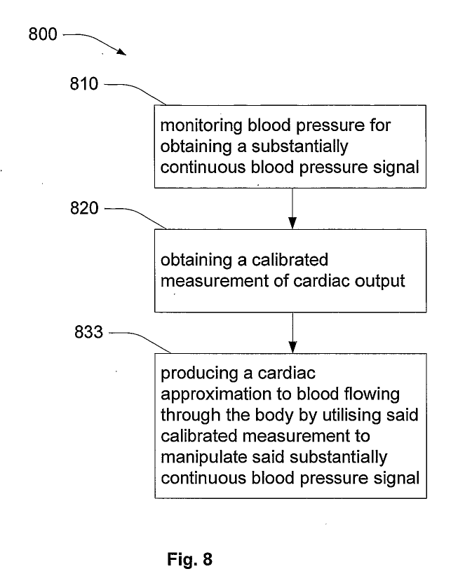

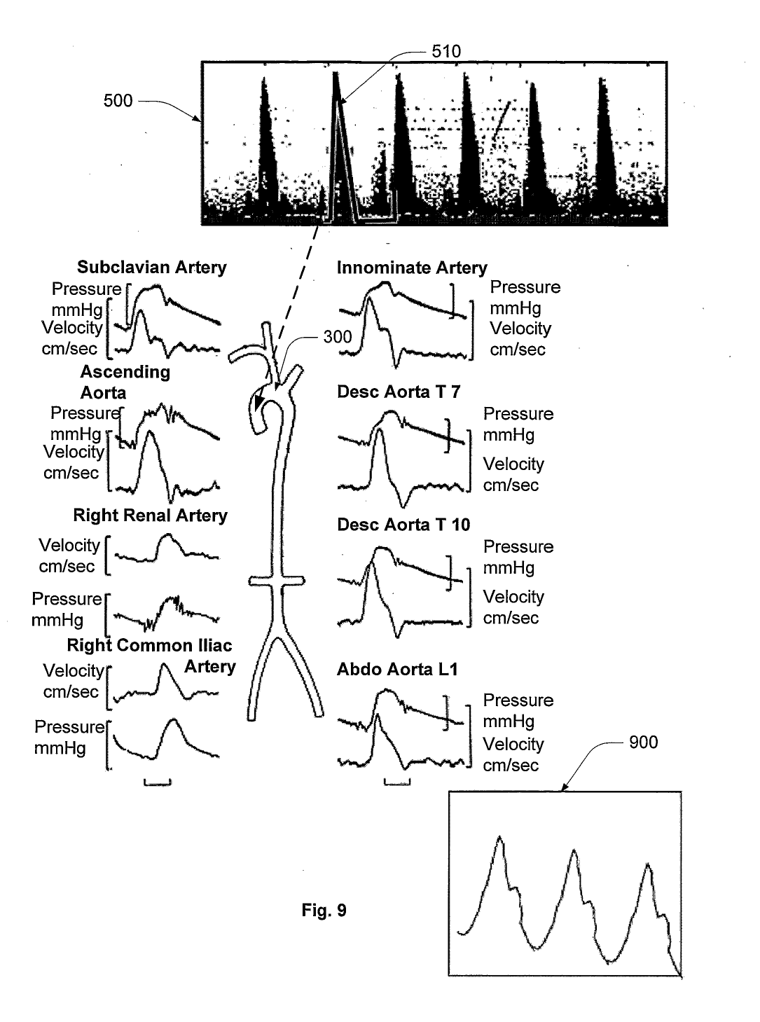

As shown in FIG. 6, there is provided a device 600 according to an embodiment comprising a first component 610 for producing a real time blood pressure signal as a non-quantitative measure of cardiac output and a second component 620 for intermittent measuring the cardiac output. A third component 630 receives the cardiac output measurement to regularly calibrate the blood pressure signal as a calibrated quantitative continuous measurement of cardiac output. This resultant device would be capable of producing a continuous calibrated measurement of cardiac output in real time.

In an example embodiment, the cardiac output monitoring device 620 is the USCOM device. The USCOM device is a non-invasive ultrasound device specialised for accurate and simple beat to beat measurement of cardiac output, but which requires intermittent measurement. This device provides a non-invasive and accurate reference measurement for calibration of a blood pressure signal, so that recalibration would be performed with relatively safely and effectively.

Embodiments utilise the USCOM device to provide a direct measure of cardiac output which is combined with a non-invasive blood pressure signal acquired by another physiologic sensor such as tonometer or sphygmomanometric signal from a pulse oximeter or a non-invasive continuous cuff blood pressure monitor or a peripheral vascular Doppler, or an invasive manometer.

A pulse oximeter provides a plethysmographic pressure wave form reflecting blood pressure and displays a real time blood pressure signal. Using this signal combined with multiple repeated recalibrations from measurements provided by the USCOM device, a non-invasive blood pressure monitoring method providing real time continuous beat to beat cardiac output is provided.

In one embodiment, the USCOM continuous wave Doppler measurement of cardiac output is used to calibrate a tonometrically generated signal from any region.

In another embodiment, the USCOM continuous wave Doppler measurement of cardiac output is used to calibrate a Doppler generated signal from any peripheral vessel.

The USCOM device provides a non-invasive cardiac output measurement method, which may be preferably combined with a non-invasive blood pressure signal measurement method to create a non-invasive continuous cardiac output monitor.

In another embodiment the USCOM continuous wave Doppler measurement of cardiac output is used to calibrate an invasively acquired blood pressure signal.

The USCOM device cardiac output measurement method can alternatively be used to calibrate an invasive blood pressure signal measurement method and create a reliable cardiac output calibration value to improve the accuracy of monitoring.

In further embodiments the USCOM device cardiac output measurement method is used in combination with an invasive blood pressure signal methods, replacing invasive calibration such as trans-pulmonary thermodilution. This reduces the relative invasiveness of these current methods.

Turning now to FIG. 7, there is illustrated, in the form of a functional block diagram, an embodiment of the processing unit 220 shown in FIG. 2. This processing unit includes a master oscillator 710 that is interconnected to a transmitter 720. The transmitter is configured to transmit the oscillation to transducer 230. A receiver 730 receives returned signals from the transducer 230, for further processing to produce a measure of cardiac output. The transducer signals are forwarded to a demodulating element 740, which utilises phase outputs from the master oscillator 710 to demodulate the tranducer signals and provide for a spectral output. This spectral output is forwarded to a spectral analyser 750. In an example embodiment, the spectral analyser 750 includes a digital signal processor (DSP) arrangement for processing the spectral output to determine a number of relevant cardiac function indicators. In other embodiments, the spectral analyser 750 can comprise a computer type device with appropriate DSP hardware. Using common statistical data processing and analysis methods on the spectral output from the demodulator, a first processing component 760 can be configured to output a number of cardiac indicators. For example these cardiac indicators include:
Heart rate;
Cardiac index (CI); the amount of blood the left ventricle ejects into the systemic circulation in one minute, measured in litres per minute;
Stroke volume (SV): the amount of blood that is put out by the left ventricle of the heart in one contraction;
Stroke volume index (SVI): the quantity of blood ejected in one cardiac cycle in millilitres per meter square per beat; and
Cardiac output (CO): the amount of blood that is pumped by the heart per unit time, measured in litres per minute, CO can be calculated by multiplying the stroke volume by the heart rate.

As the master oscillator 710 is able to provide a continuous signal to the transmitter 720 and hence the transducer 230, a continuous flow signal is provided to the receiver 730 and demodulator 740. This enables the cardiac indicators to be continuously updated, and hence provide real-time monitoring of cardiac indicators on a beat-to-beat basis. The real-time monitoring of cardiac indicators is commonly used for detecting and determining severity of cardiovascular disease and guiding therapy

A blood pressure signal is provided by the blood pressure sensor 240 to a second processing component 770. The blood pressure signal provided the systolic and diastolic blood pressure values which allows the second processing component 770 to calculate additional cardiac indicators. For example these additional cardiac indicators include:
Mean arterial pressure (MAP): the average pressure within an artery over a complete cycle of one heartbeat;
Systemic vascular resistance (SVR): an index of arteriolar constriction throughout the body, calculated by dividing the blood pressure by the cardiac output;
Cardiac Stroke Work (SW): the amount of energy that is given to the blood in a single heart stroke;
Cardiac Power Output (CPO): the product of mean Blood Pressure (BP) and Cardiac Output (CO).

In an embodiment, the availability of blood pressure measurements allows calculation of cardiac stroke work (SW) that is used to assess ventricular function. The SW value is obtained by combining the cardiac power output (CPO) with the flow time measured from the transducer to obtain an energy value for a single beat. The typical units of SW are milli-Joules. An example formula used to calculate SW is:

$${SW}_{m J} = \frac{60}{450} ( {MAP}_{m mHg} - {CVP}_{m mHg} ) \cdot {SV}_{m l} ,$$

where CVP is the central venous pressure which is determined from pressure measurements from the blood pressure sensor 240. CVP is preferably included for improved accuracy, but is commonly unavailable and excluded from the calculation.

If SW is plotted against ventricular preload, the resulting ventricular function curve appears qualitatively similar to a Frank-Starling curve. Like the Frank-Starling relationship, there can be a family of curves depending upon the inotropic state of the ventricle. SW is also proportional to MVO₂ (Myocardial Oxygen Consumption) and usually indexed by the patient's body surface area (BSA) to give the stroke work index as defined by SWI=SW/BSA.

In an embodiment, Cardiac Power output (CPO) can be presented as another important cardiac indicator in the monitoring of cardiac function and diagnosis of dysfunction. CPO can be calculated using the formula

CPO_{Watts}=(MAP_{mmHg}−CVP_{mmHg})·CO_{1.1 min}(kg·m²s⁻³),

where (MAP_{mmHg}−CVP_{mmHg}) can be approximated by the patient's blood pressure obtained from the blood pressure sensor 240, CO is the patient's cardiac output obtained from the transducer 230 in units of litre per minute. CVP is preferably included for improved accuracy, but is commonly unavailable and excluded from the calculation. The result can be divided by a scaling factor of approximately 450.037 to give the result is in units of power (Watts).

The above system of conducting cardiac power measurements may be incorporated into an automatic heart monitoring system so as to provide continuous, beat to beat, cardiac power measurements. Such a system can be a suitably reprogrammed version of the system proposed in PCT Patent Application No: PCT/AU2006/000338 filed 15 Mar., 2006, entitled “Automatic Flow Tracking System and Method”, the contents of which are hereby incorporated by reference.

FIG. 7 shows a flowchart of method 700 according to an embodiment, this method comprising the steps of:
a) performing a substantially continuous blood pressure monitoring at a first predetermined location to obtain a substantially continuous blood pressure signal 710;
b) obtaining a calibrated measurement of cardiac output 720 from a direct cardiac output device; and
c) produce a cardiac approximation to blood flowing through the body by utilising the calibrated measurement to manipulate the substantially continuous blood pressure signal 730.

### FURTHER INTERPRETATION

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

Unless specifically stated otherwise, as apparent from the following discussions, it is appreciated that throughout the specification discussions utilizing terms such as “processing,” “computing,” “calculating,” “determining”, “applying” or the like, refer to the action and/or processes of a computer or computing system, or similar electronic computing device, that manipulate and/or transform data represented as physical, such as electronic, quantities into other data similarly represented as physical quantities.

It will be understood that the steps of methods discussed are performed in one embodiment by an appropriate processor (or processors) of a processing (i.e., computer) system executing instructions (computer-readable code) stored in storage. It will also be understood that the invention is not limited to any particular implementation or programming technique and that the invention may be implemented using any appropriate techniques for implementing the functionality described herein. The invention is not limited to any particular programming language or operating system.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

It would be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

The foregoing describes forms of the present invention. Modifications, obvious to these skilled in the art can be made thereto without departing from the scope of the invention.

1. A method for monitoring blood flows through a patient, the method comprising:
(a) performing a substantially continuous blood pressure monitoring of a vessel at a first predetermined location to obtain a substantially continuous blood pressure signal;
(b) performing blood flow monitoring of a vessel at a first predetermined location to obtain a substantially continuous blood flow signal;
(c) providing an updated cardiac measure of said patient by continuously processing said flow signal and said pressure signal.

2. A method according to claim 1 wherein said updated cardiac measure is a measure of Cardiac Power Output (CPO).

3. A method according to claim 2 wherein said blood pressure signal is processed to provide a measure of Mean Arterial Pressure (MAP), said blood flow signal is processed to provide a measure of Cardiac Output (CO), and said Cardiac Power Output (CPO) is substantially calculated using the formula CPO_{Watts}=(MAP_{mmHg}−CVP_{mmHg})·CO_{1.1 min}(kg·m²s⁻³).

4. A method according to claim 1 wherein said updated cardiac measure is a measure of Stroke Work (SW).

5. A method according to claim 4 wherein said blood pressure signal is processed to provide a measure of Mean Arterial Pressure (MAP), said blood flow signal is processed to provide a measure of Stroke Volume (SV), and said Cardiac Work (CW) is substantially calculated using the formula
$${SW}_{m J} = \frac{60}{450} ( {MAP}_{m mHg} - {CVP}_{m mHg} ) \cdot {SV}_{m l}$$

6. A method according to claim 1 wherein said updated cardiac measure is a measure of Cardiac Work (CW).

7. A method according to claim 1 wherein said updated cardiac measure is a substantially continuous measure of blood flowing through said patient calculated by utilising said blood flow signal to provide an updated cardiac output measurement and calibrating said substantially continuous blood pressure signal.

8. A method according to claim 7, comprises the steps of:
(a) calculating a transfer function for transforming said substantially continuous blood pressure signal to produce said substantially continuous measure of blood flowing; and
(b) producing said substantially continuous measure of blood flowing by applying said transfer function to said substantially continuous blood flow signal.

9. A method as claimed in claim 8, wherein said transfer function is a scalar value.

10. A method according to claim 1, wherein said flow signal is provided by a continuous wave Doppler ultrasonic transducer.

11. A method according to claim 10 wherein said flow signal is processed to provide beat-to-beat measures of Cardiac Output (CO) or Stroke Volume (SV)

12. A method as claimed in claim 1 wherein said pressure signal is provided by a blood pressure monitor.

13. A method as claimed in claim 1 wherein said cardiac measure is updated repeatedly and automatically.

14. A system for monitoring blood flows through a patient, the system comprising:
a transducer element for providing a blood flow signal;
a blood pressure monitor for providing a blood pressure signal;
a processing unit adapted to receive said flow signal and said pressure signal, and configured to process said flow signal and said pressure signal for providing an updated cardiac measure of said patient.

15. A system according to claim 14 wherein said transducer element includes a continuous wave Doppler ultrasonic transducer.

16. A system according to claim 14, wherein said blood flow signal is provided by a USCOM device.

17. A system according to claim 14 wherein said cardiac measure is Cardiac Power Output (CPO).

18. A system according to claim 17 wherein said flow signal and said pressure signal are each substantially continuous and said Cardiac Power Output is provided substantially in real-time.

19. A system according to claim 14 wherein said flow signal and said pressure signal is processed to provide beat-to-beat measures of any one of Cardiac Power Output (CPO), Stroke Work (SV), or Cardiac Work (CW).

20. A system according to claim 14 wherein said processing unit adapted to applying a transfer function to a substantially continuous said blood flow signal for producing said cardiac measure in the form of a substantially continuous measure of blood flowing through said patient.
